(19) United States
(12) Patent Application Publication
(10) Pub. No.: US 2006/0190057 A1
(43) Pub. Date: Aug. 24, 2006

(54) Synthetic traveling wave transcutaneous electrical stimulation device

(76) Inventor: Edward W. Reese, Prior Lake, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 11/065,234

(22) Filed: Feb. 24, 2005

(51) Int. Cl. A61N 1/34

(52) U.S. Cl. 607/46

(57) ABSTRACT

A transcutaneous electrical nerve stimulation device for treating a user's lower back pain or discomfort is provided. The inventive device comprises a stimulation pad with an array of electrodes therein. A controller communicates with the electrodes to energize/de-energize them in a patterned, sequential manner. The patterned actuation of the electrodes in a sequential fashion provides electrical impulses to the patient's skin, simulating a transverse wave motion. The transverse wave motion simulation provides the user with the effect of the electrical stimulation combined with the mechanical tissue stimulation received in traditional massage therapy, resulting in improved pain and discomfort relief. The device may use programs stored in the controller. Alternatively, the user may manually actuate one or more electrodes as well as manually move the electrode actuation to concentrate on a specific lower back region using an electrostatic touch pad or the equivalent. The user may also use voice commands to access the stored programs, move the electrode stimulation region, change the electrode actuation intensity and/or dwell time. The device may store the user's manually-generated routines for repeating when desired. A visual display may provide feedback to the user regarding the electrode actuation position and pattern.

### FIELD OF THE INVENTION

The present invention is directed generally to medical devices and more particularly to a device for treating lower back pains or discomfort using transcutaneous electrical nerve stimulation with an adjunctive propagating motion that generates an additional massage-like therapeutic effect.

### BACKGROUND

Transcutaneous electrical nerve stimulation, commonly referred to as TENS, has been widely accepted for some time now as an effective means for treating both acute and chronic pain in human patients. Devices of this type generally include a method of producing electrical pulses and one or more electrodes in direct contact with the patient's skin in the area to be treated. In practice, the applied electrical pulses to the patient's skin generate a small current through the patient's underlying tissue near the electrode(s) which ultimately produce the therapeutic effect. Various geometrical configurations have been developed for the electrodes which conform to the particular body part to be treated, e.g., wrist, forearm, upper leg region, lower back, etc. Also, most devices have given the patient some flexibility to adjust both the strength and duration of the electrical pulses to customize the procedure to the patient's liking.

Combination devices have also been developed which deliver both transcutaneous electrical nerve stimulation and heat (via infrared light sources) to the same tissue region via integrated electrical/optical electrode configurations. However, none of the prior art devices have incorporated a method to integrate the therapeutic action of the TENS device with the soothing response received in a traditional massage with mechanical tissue stimulation coupled with simultaneous transverse motion.

The present invention addresses these problems

### SUMMARY OF THE INVENTION

A transcutaneous electrical nerve stimulation device for treating a user's lower back pain or discomfort is provided. The inventive device comprises a stimulation pad with an array of electrodes therein. A controller communicates with the electrodes to energize/de-energize them in a patterned, sequential manner. The patterned actuation of the electrodes in a sequential fashion provides electrical impulses to the patient's skin, simulating a transverse wave motion. The transverse wave motion simulation provides the user with the effect of the electrical stimulation combined with the mechanical tissue stimulation received in traditional massage therapy, resulting in improved pain and discomfort relief. The device may use programs stored in the controller. Alternatively, the user may manually actuate one or more electrodes as well as manually move the electrode actuation to concentrate on a specific lower back region using an electrostatic touch pad or the equivalent. The user may also use voice commands to access the stored programs, move the electrode stimulation region, change the electrode actuation intensity and/or dwell time. The device may store the user's manually-generated routines for repeating when desired. A visual display may provide feedback to the user regarding the electrode actuation position and pattern.

An object of the inventive device and method is to provide a TENS unit that combines electrical stimulation with simultaneous transverse motion that simulates a traveling wave of stimulation.

An object of the inventive device and method is to provide a TENS unit that allows simulation of waves of stimulation via programs stored within the device.

Another object of the inventive device and method is to provide a TENS unit that allows the user to manually move the electrode actuation point to a specific region of pain or discomfort using a controller.

Still another object of the inventive device and method is to provide a TENS unit allows the user to use voice commands to move the electrode actuation point to a specific region of pain or discomfort and modify the electrode actuation intensity and dwell time.

Yet another object of the inventive device and method is to provide a TENS unit that allows user programming of electrode actuation patterns, locations, intensity and dwell time.

Still another object of the inventive device and method is to provide a TENS unit that provides the user with visual feedback of the electrode actuation location and pattern.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 depicts an artist's rendition of an embodiment of the transcutaneous electrical nerve stimulation device mounted in the lower back region of a patient and bi-directional vertically moving waves of transcutaneous electrical nerve stimulation.

FIG. 1A provides an illustration of one embodiment of the controller device.

FIG. 1B provides an illustration of a user wearing the device and interacting with a wireless controller.

FIG. 1C illustrates a user interacting with a controller mounted on a belt.

FIG. 2 shows one embodiment of an electrode array matrix and the time domain sequence capable of generating bi-directional vertically moving waves of transcutaneous electrical nerve stimulation.

FIG. 2A provides a block diagram for generating bi-directional vertically moving waves transcutaneous electrical nerve stimulation.

FIG. 3 depicts an artist's rendition of an embodiment of the transcutaneous electrical nerve stimulation device mounted in the lower back region of a patient and bi-directional horizontally moving waves of transcutaneous electrical nerve stimulation.

FIG. 4 shows one embodiment of the electrode array matrix and the time domain sequence capable of generating bi-directional horizontally moving waves of transcutaneous electrical nerve stimulation.

FIG. 4A outlines a block diagram for generating bi-directional horizontally moving waves of transcutaneous electrical nerve stimulation

FIG. 5 provides an artist's rendition of an embodiment of the transcutaneous electrical nerve stimulation device mounted in the lower back region of a patient and radial burst waves of transcutaneous electrical nerve stimulation.

FIG. 6 shows one embodiment of the electrode array matrix and the time domain sequence capable of generating a radial burst waves of transcutaneous electrical nerve stimulation.

FIG. 6A provides a block diagram for generating radial burst waves of transcutaneous electrical nerve stimulation.

FIG. 7 provides an artist's rendition of an embodiment of the transcutaneous electrical nerve stimulation device mounted in the lower back region of a patient and bi-directional vertically and horizontal moving waves of transcutaneous electrical nerve stimulation.

FIG. 8 provides an artist's rendition of an embodiment of the transcutaneous electrical nerve stimulation device mounted in the lower back region of a patient and circularly moving waves of transcutaneous electrical nerve stimulation.

FIG. 9 provides one embodiment of the electrode array matrix and the time domain sequence capable of generating a circularly moving wave of transcutaneous electrical nerve stimulation.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

In general the present invention is directed to medical devices and more particularly to a device for treating lower back pain with transcutaneous electrical nerve stimulation with an adjunctive propagating motion that may simulate a traveling wave and that generates an additional massage-like therapeutic effect.

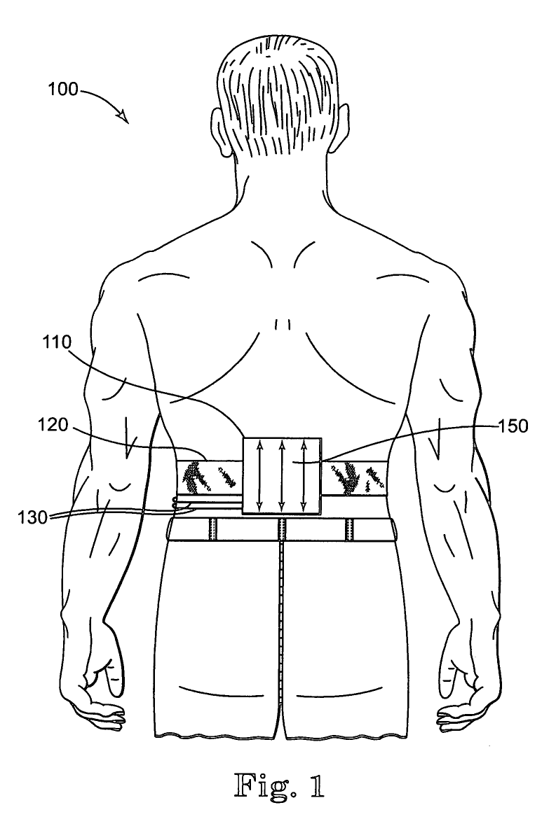

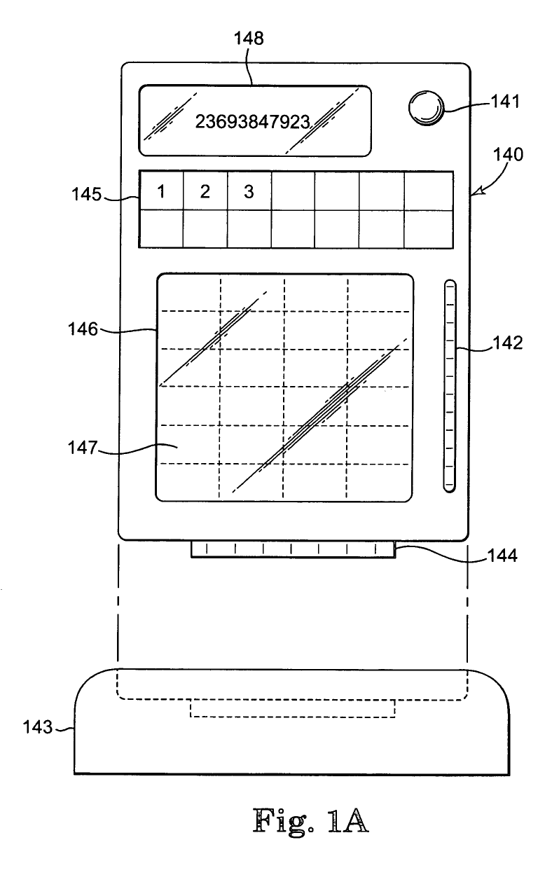

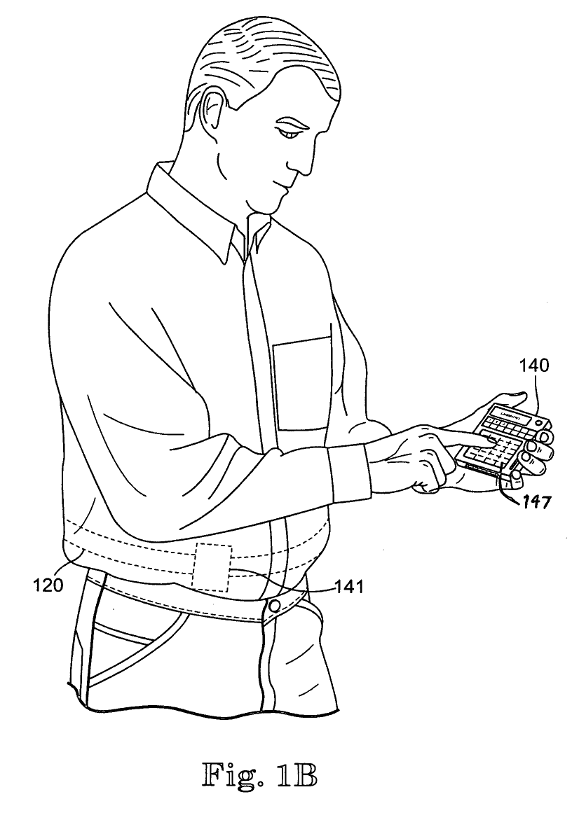

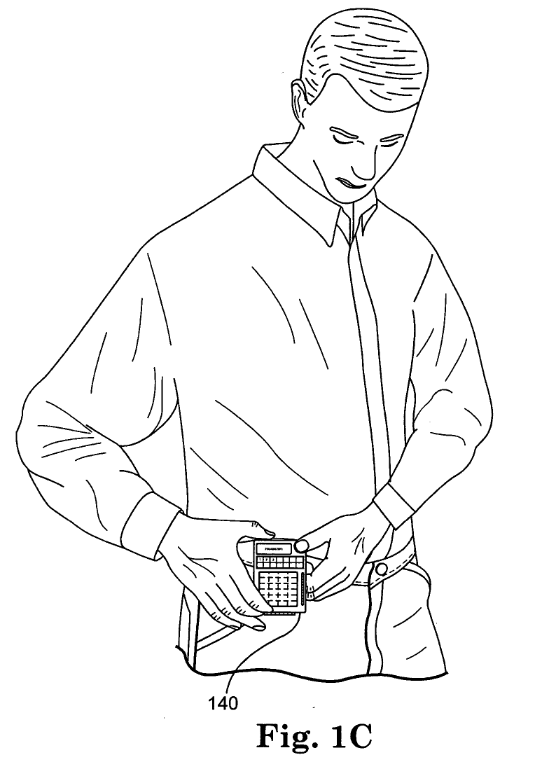

With reference to the Figures, one embodiment of a transcutaneous electrical nerve stimulation unit 100 with an adjunctive propagating motion is depicted in FIG. 1. A stimulation pad 110 may be placed in direct contact with the patient's lower back region and may be held in place by a belt-like fastener 120. Equivalent devices and methods for contacting the stimulation pad will readily present themselves to those skilled in the art. The stimulation pad 110 may comprise a pad that surrounds or encloses or encases an array of a plurality of electrodes that may be arranged in a pattern. For example, a two-dimensional matrix array of a plurality of electrodes maybe be used wherein the electrodes may be individually energized for transcutaneous electrical nerve stimulation. The plurality of electrodes arranged in a pattern may comprise a flex circuit, though other equivalent alternatives are well known to those skilled in the art. The stimulation pad's plurality of electrodes may communicate in wired or wireless fashion with a controller and/or a transceiver. Lead wires 130 indicate one wired communication embodiment.

FIG. 1 further provides one illustrative embodiment of a synthetic traveling wave of stimulation program, algorithm or routine that the inventive device is capable of producing. The wave of stimulation shown is a bi-directional vertical traveling wave 150 and will be discussed in greater detail infra.

FIG. 1A illustrates one embodiment of the controller 140. The front panel of the controller 140 may have, inter alia, a power button or switch 141 to activate/deactivate the TENS device and a battery charge level indicator 142 to indicate the charge level for on-board batteries. The controller 140 may have a stand-alone docking station 143 that may be used to access power and/or to recharge the on-board batteries. As illustrated, one embodiment for the docking station 143 provides an electrical connector 144 on the controller 140 whereby the controller 140 plugs into or operatively connects with the docking station 143.

The controller 140 may also provide for keypad entry 145 wherein the user may select pre-programmed TENS stimulation routines or, alternatively, the user may choose to operate the TENS unit in manual mode, by utilizing the user-controlled electrode actuator 147. In the manual mode, the user may manually energize and de-energize one or more of the electrodes through the electrode actuator. The controller 140 may further comprise a processor and a memory for storing pre-programmed routines or for capturing manually generated routines. Moreover, the controller may comprise devices for communicating wirelessly with the plurality of electrodes, either directly with the electrodes or via a transceiver. Such wireless communication mechanisms are well known in the art and are not shown in the Figures.

Various embodiments of the electrode actuator comprise an electrostatic touchpad 147 as shown in FIGS. 1A and 1B and further wherein the touchpad presents a scaled correspondence with the patterned array of the plurality of electrodes within the stimulation pad as illustrated by the grid lines for the touch pad 147. In this embodiment, each grid may be presented in scaled correspondence with one of the plurality of electrodes within the matrix or pattern. To use the touchpad 147 as a manual electrode actuator, the user may, e.g., manually locate or center the electrode actuation point or energizing/de-energizing point over a specific region of pain or discomfort of the lower back. This may be accomplished by the user touching at least one grid with a finger or pen or the equivalent on the touchpad 147 as suggested in FIG. 1B. Touching a specific grid may actuate the correspondent electrode within the electrode array for a default dwell time and intensity, resulting in a single point/electrode stimulation over the selected lower back region. The user may also modify the electrode actuation intensity and dwell time using the keypad 145 or equivalent mechanism. Sliding the touch pen, or the user's finger, across the grids on the touchpad 147 may result in actuation of the correspondent electrodes in the electrode array in sequential fashion or pattern. This provides, in turn, stimulation across the user's lower back reflecting the same ordered pattern as the path of the touch pen or user's finger.

In this manner, the user may locate the electrode actuation over a specific region of lower back pain and may manually stimulate the specified area using a manually generated stimulation routine. Moreover, the user may wish to store this manually located electrode actuation position and manually generated stimulation routine within the controller's memory for later use.

Other embodiments of a manual electrode actuator for locating the stimulation over a certain point of the lower back and generating a manual stimulation routine readily present themselves to those skilled in the art. For example, a gaming-type joystick may be used whereby the user directionally manipulates the joystick to move within the plurality of electrode pattern and actuate or energize individual electrodes. Still other embodiments may include simple directional (up/down and left/right) buttons to effect movement within a two-dimensional matrix array of electrodes, or a non-scaled electrostatic pad such as that used on laptop and notebook personal computers to move the cursor.

Other embodiments of the inventive device may utilize voice activation as a “manual” electrode actuator whereby the controller 140 uses mechanisms well known within the art to receive, and act upon, voice commands to move the electrode actuation across the patterned plurality of electrodes. For example, a user may initiate a manual voice-activated mode, wherein the electrode actuation may begin pulsing in a generally central location, e.g., within a centralized region of the matrix of electrodes. The user may wish to move the electrode actuation to a specific point of discomfort on the lower back and, using the voice activation feature, may issue vocal commands, e.g., “left” wherein the electrode actuation moves one matrix element to the left, or “up” or “down” wherein the electrode actuation moves up or down, respectively. In addition, the user may modify the stimulation intensity and/or dwell time using specific voice commands.

As described above, the voice-activated electrode actuation point and voice-activated electrode actuation routine may be stored within the controller's memory. The routine may then be run at a later time.

To aid in the manual or voice-activated manipulation of the electrode actuation locating and stimulating features described above, a visual feedback may be provided to the user via the touch pad 147. This visual feedback feature may also be provided when operating in the pre-programmed stimulation mode. In this embodiment, the touch pad 147 may also function as a feedback display wherein visual indication of the position of the electrode actuation is provided, or a separate visual feedback display provided either mounted on the controller or outputted to a monitor such as a computer monitor. Such feedback may be represented by a flashing light or other equivalent means to represent the electrode stimulation point at any given time during the process of locating the electrode actuation over a specific lower back region or during the execution of a pre-programmed or stored routine. In this way, a user may more accurately manually move the electrode actuation to position it over the point of discomfort on the lower back more easily. Moreover, the user may receive visual feedback about the programmed or manual sequencing of electrode actuation that is occurring.

As discussed above, the electrical leads 130 for the plurality of electrodes within the stimulation pad 110 illustrated in FIG. 1 may be directly connected to the controller 140 in a wired communication embodiment. Alternatively, the controller 140 may be connected to the plurality of electrodes within the stimulation pad 110 in a wireless communication embodiment. Thus, as indicated in FIGS. 1B and 1C, the controller 140 may be hand-held or detachably attached to the user's belt-like fastener 120.

FIG. 1B illustrates a user interacting with the control unit 140 that is in wireless communication with still another alternative embodiment comprising a transceiver 141 supportably attached to the belt-like fastener 120, shown worn beneath the user's clothing in contact with the user's skin to allow the user to operate the TENS unit in an inconspicuous manner while at work or other public place. The transceiver 141 is in wireless communication with the controller 140 to receive either pre-programmed, stored or manual electrode actuation signals from the controller 140. The electrode actuation signals comprise the commands to energize/de-energize selected electrodes within the plurality of electrodes, the pattern of the electrode actuation, as well as the actuation intensity and dwell time. The transceiver 141, in turn, transmits the electrode actuation signals in either a wired or wireless communication to the plurality of electrodes.

As further suggested by FIG. 1B, the user may input commands to the TENS device by keystroking the controller 140, or in environments where it may be necessary for totally hands-free usage, e.g., while driving a car, the controller 140 may be designed to incorporate well-known voice activation technology whereby the controller 140 accepts voice commands and responds accordingly.

Alternate embodiments of the inventive device may combine the synthetic traveling wave abilities described above with additional stimulation therapies. For example, the plurality of electrodes, or components of the flex circuit or wires therein contained within the stimulation pad may be designed with the capability to adjustably warm or heat the pad surface in a manner well known to those skilled in the art using, e.g., heating elements. The combination of the traveling wave therapy with heat may result in increased, even synergistic, effects on the user's lower back pain or discomfort. The variation in temperature may be programmable in combination with the traveling wave stimulation programs described above. Moreover, discrete zones within the stimulation pad may be programmed for differential heating treatments.

Moreover, another therapy that may be readily incorporated into the inventive device may be vibration, stimulation well known by those skilled in the art. For example, the user may wish to have all, or a portion thereof, of the stimulation pad to vibrate while receiving a traveling wave stimulation as described above. Such vibration elements may be programmable along with either the traveling wave stimulation alone or in combination with the heating capability described above. As above, such programmable vibration may be achieved throughout the stimulation pad, or within discrete zones of the pad.

The device having thusly been described, an exemplary approach to programming algorithms or routines as software code within the controller's processor will now be described.

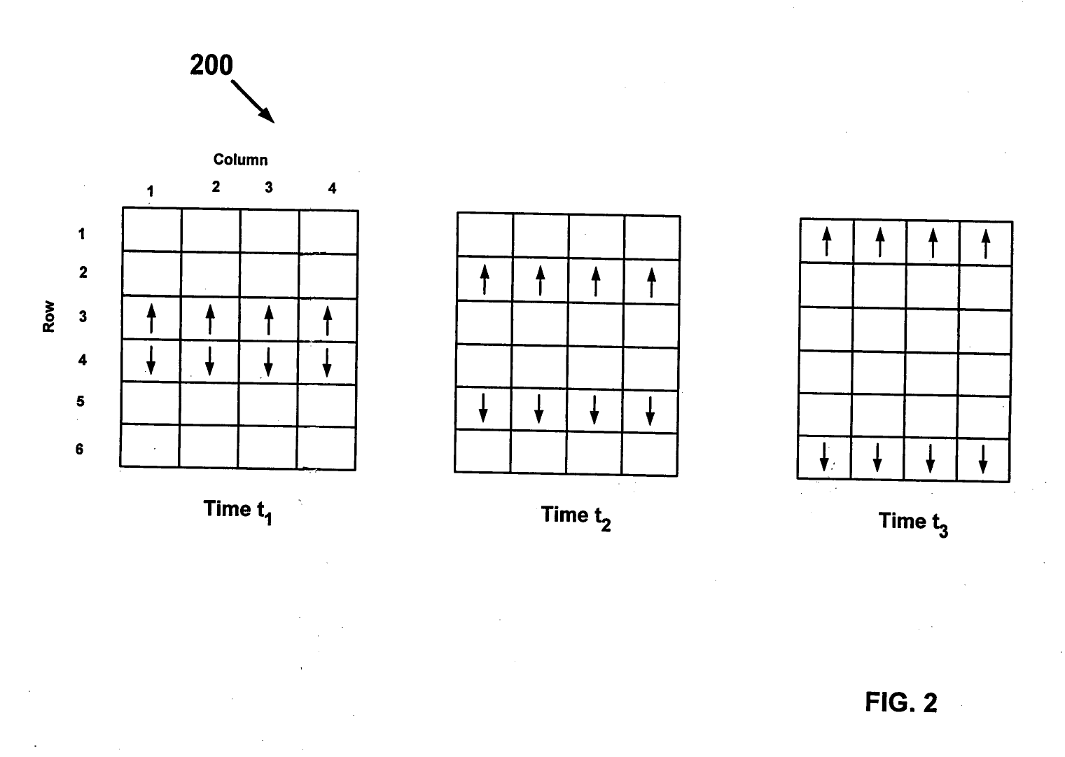

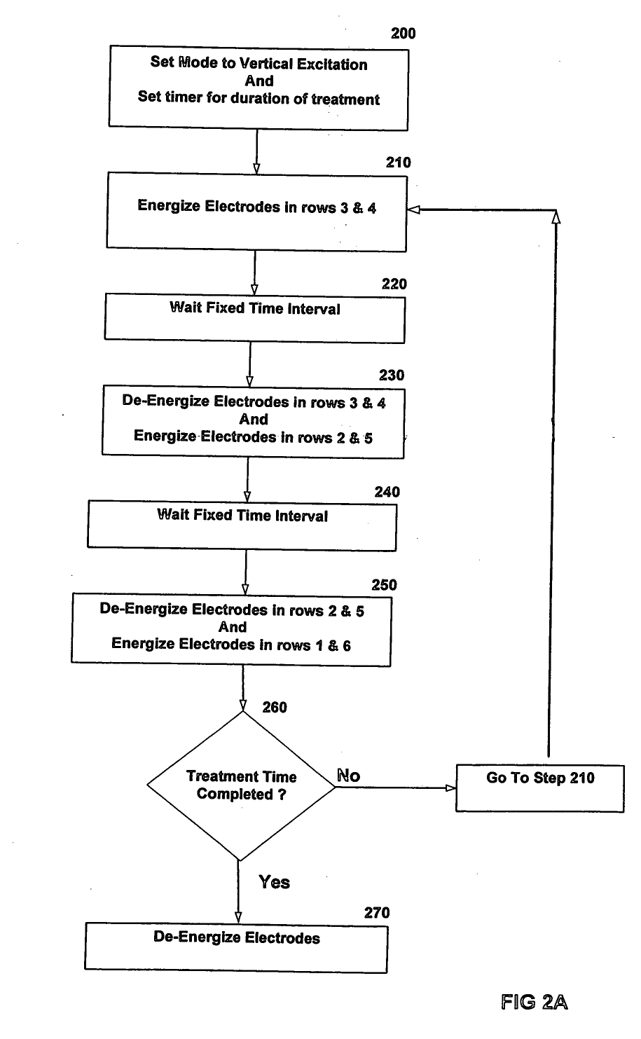

FIG. 2 shows one embodiment and illustration of the matrix array of electrodes 200 arranged within the stimulation pad. The traveling sequential pattern illustrated in FIG. 2 corresponds with the bi-directional vertical traveling wave motion described above in connection with FIG. 1.

FIG. 2 schematically depicts a 6 row by 4 column matrix array of individual electrode pairs 200 which may be individually energized. The size of the actual matrix employed herein is for illustrative purposes only and may be larger or smaller in practice.

As shown at time t₁, rows 3 and 4 may be energized generating a localized transcutaneous electrical nerve stimulation near the central region of the matrix. A short time later at time t₂, rows 3 & 4 may be de-energized substantially synchronous with the energizing of rows 2 & 5, this localized transition may give the patient a sensation that the TENS signal is radiating (traveling) in a bi-directional vertical manner, yielding a simultaneous up & down wave motion. And finally, a short time later at time t₃, the outermost row of electrodes, rows 1 & 6, may be energized reinforcing the sensation that the TENS signal is traveling further away from the central region of the matrix. The above spatial (electrode matrix stimulation) and temporal (at repetitive times t₁, t₂, and t₃) can be repeated over-and-over again to yield a soothing massage-like traveling wave sensation along with the transcutaneous electrical nerve stimulation.

It will be apparent to those skilled in the art that use of such matrix-based algorithms provide a virtually limitless number of programs or routines that may be developed and implemented using the invention described herein; each such program or routine is within the scope of the invention. For example, the above-described bi-directional vertically traveling waves begin at the center of the electrode matrix array, radiate upward and downward respectively, then return to the starting point and repeat the sequence. Alternative embodiments may include reversing the algorithm when it reaches the electrodes on the outer matrices of the pad so that the traveling wave reverses and moves toward the center. Alternatively, the traveling waves may be initiated at t₁ only in column 1, with subsequent waves initiating at t₂ in adjacent columns. Similarly, the waves may initiate in the center column(s) at t₁, with subsequent waves beginning at time t₂, t₃, etc. in the next outer adjacent column to simulate a traveling wave occurring in both the vertical and horizontal dimensions.

The vertical TENS signal routine outlined above may, as described above, be pre-programmed in the controller and initiated by accessing the program menu either by keystroking the controller keys or other equivalent manual actuation mechanism or by issuing vocal commands that are translated by the device's voice-activated capabilities.

FIG. 2A depicts a block diagram illustrative of the algorithm that may be embedded in the controller unit 140 to execute a bi-directional vertical TENS program. In block 200, the user may select the vertical excitation up & down mode and select an auto-stop time interval. The user may then actuate the start button and the electrodes in rows 3 and 4 will be energized 210. The algorithm may be programmed to dwell a predetermined time interval 220 with electrodes 3 and 4 energized. Once the predetermined time interval has elapsed, the controller unit 140 may initiate a command to de-energize electrodes 3 and 4 while energizing electrodes 2 and 5 (230). As before, the algorithm may be programmed to dwell a predetermined time interval 240 with electrodes 2 and 5 energized. Once the predetermined time interval set in 240 has elapsed, the controller unit 140 may initiate a command to de-energize electrodes 2 and 5 while energizing electrodes 1 and 6 (250). After energizing electrodes 1 and 6 for a preset time interval, the controller unit 140 may compare the program run time with the user set treatment time in line 260, and if the run time exceeds the user set treatment time, the controller unit 140 may terminate the session by de-energizing the electrodes 270, otherwise the program may loop back to line 210 of the code and continue to run until eventually timing out in line 260.

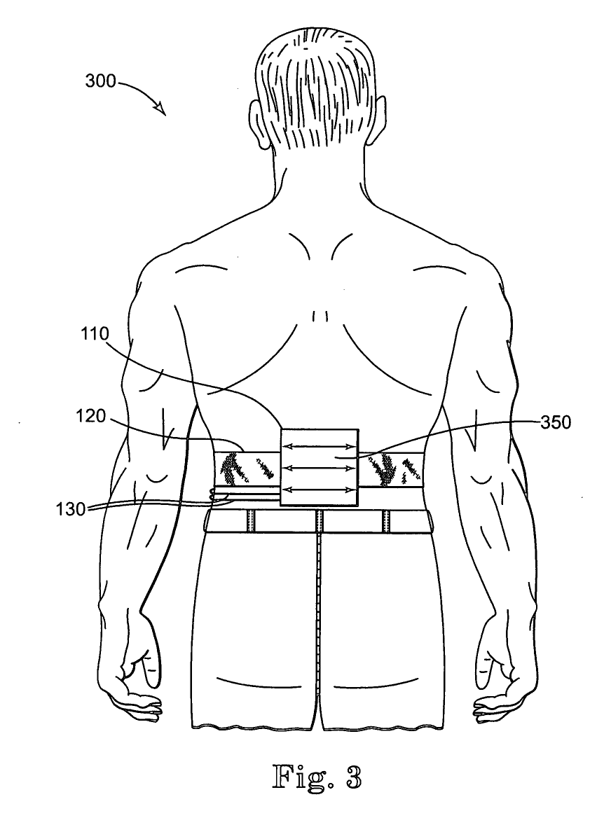

Another embodiment of a transcutaneous electrical nerve stimulation algorithm, program or routine is illustrated in FIG. 3 showing the stimulation pad 110 secured by the belt-like fastener 120 as described above. In this embodiment, energizing individual electrodes in the matrix array may allow for a traveling wave sensation for the patient such as the bi-directional horizontal motion 350 depicted in FIG. 3.

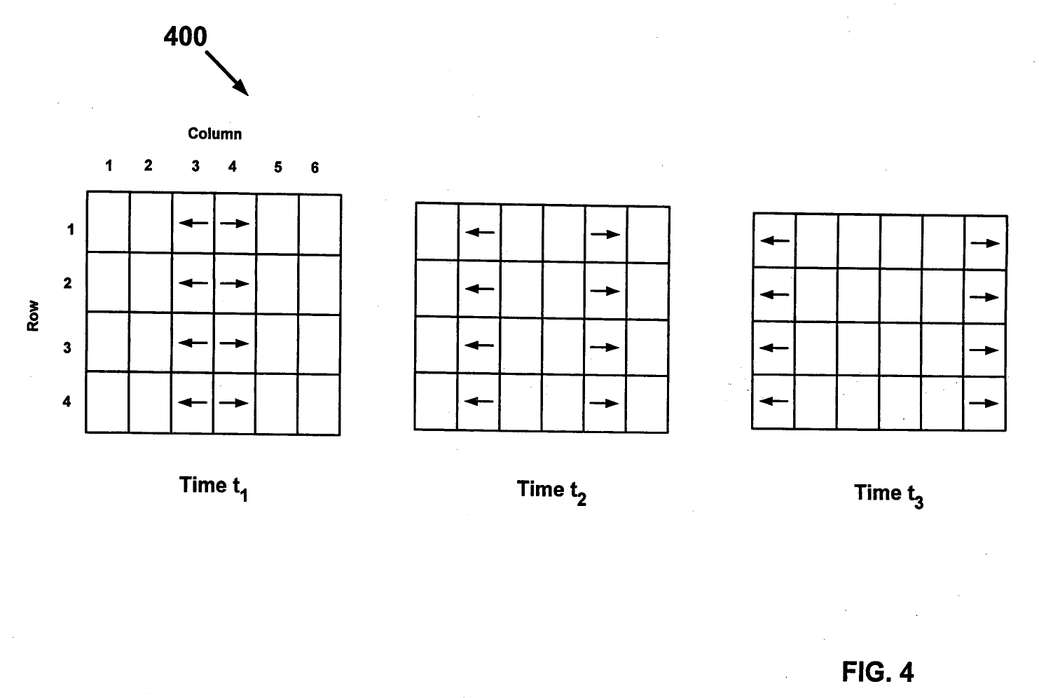

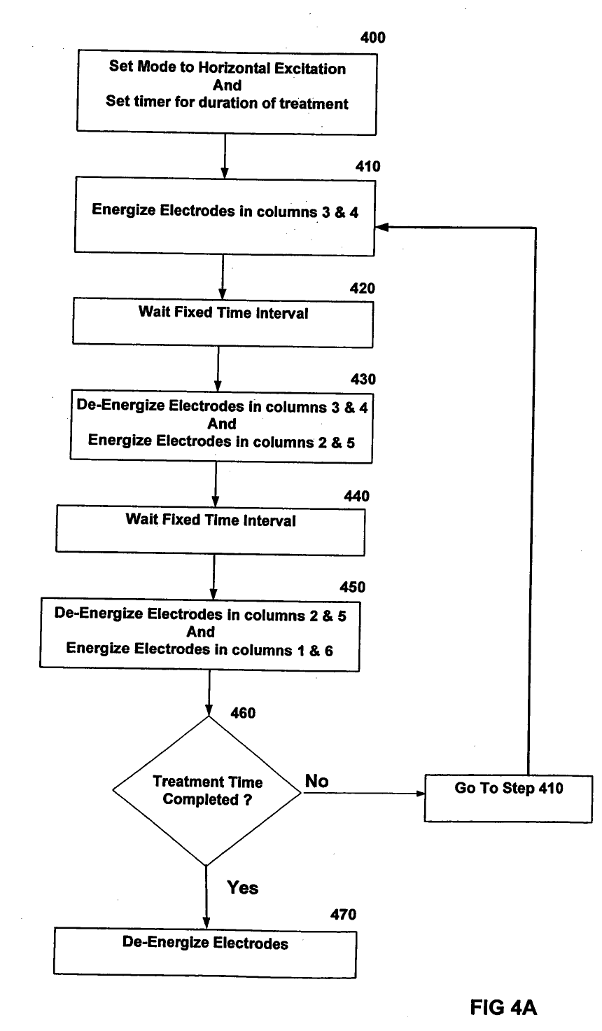

FIG. 4 shows one embodiment of the matrix array of electrodes 400 capable of generating the bi-directional horizontal motion 350 of FIG. 3. In this embodiment, the matrix array of electrodes 400 is shown schematically as a 4 row by 6 column array of individual electrodes which may be individually energized. Again, the matrix size is for illustrative purpose only and is not limiting. As shown at time t₁, columns 3 and 4 may be energized generating a localized transcutaneous electrical nerve stimulation near the central region of the matrix. A short time later at time t₂, columns 3 & 4 may be de-energized substantially synchronous with the energizing of columns 2 & 5, this localized transition may give the patient a sensation that the TENS signal is radiating (traveling) bi-directionally horizontally outward from the center of the matrix. And finally, a short time later at time t₃, the outermost columns of electrodes, columns 1 & 6, may be energized reinforcing the sensation that the TENS signal is traveling further away from the central region of the matrix. The above spatial (electrode matrix stimulation) and temporal (at repetitive times t₁, t₂, and t₃) may be repeated over-and-over again to yield a soothing massage-like traveling wave sensation along with the transcutaneous electrical nerve stimulation.

As described above, this is simply one exemplary program, algorithm or routine that may be programmed into the controller; others will readily present themselves to those skilled in the art and, therefore, are within the scope of the present invention. Moreover, as with all possible programs, algorithms or routines, the bi-directional horizontal TENS signal outlined above may be pre-programmed in the controller and initiated by accessing the program menu either manually or by voice-activated command.

FIG. 4A illustrates a block diagram of the program, algorithm or routine that may be embedded in the controller to execute the bi-directional horizontal TENS signal routine of FIGS. 3 and 4. In block 400, the user may select the horizontal excitation mode and select an auto-stop time interval. The user may then actuate the start button and the electrodes in columns 3 and 4 will be energized 410. The controller may be programmed to dwell a predetermined time interval 420 with electrodes in columns 3 and 4 energized. Once the predetermined time interval has elapsed, the controller may initiate a command to de-energize electrodes in columns 3 and 4 while energizing electrodes in columns 2 and 5 (430).

The controller may be programmed to dwell a predetermined time interval 440 with electrodes in columns 2 and 5 energized. Once the predetermined time interval set in block 440 has elapsed, the controller may initiate a command to de-energize electrodes in columns 2 and 5 while energizing electrodes in columns 1 and 6 (450). After energizing electrodes in columns 1 and 6 for a preset time interval, the controller may compare the program run time with the user set treatment time in line 460, and if the run time exceeds the user set treatment time, the controller may terminate the session by de-energizing the electrodes 470, otherwise the program may loop back to block 410 and continue to run until eventually timing out 460.

The astute reader will appreciate that a similar result could have been attained as shown in FIG. 3 by simply rotating the stimulation pad 110 shown in FIG. 1 by 90 degrees in either the clockwise or counter-clockwise direction. Alternatively, the stimulation pad may be rotated 45 degrees so that the stimulation appears to travel in a diagonal fashion. The stimulation pad 110 may be designed to rotate to any desired angular orientation that the user may choose.

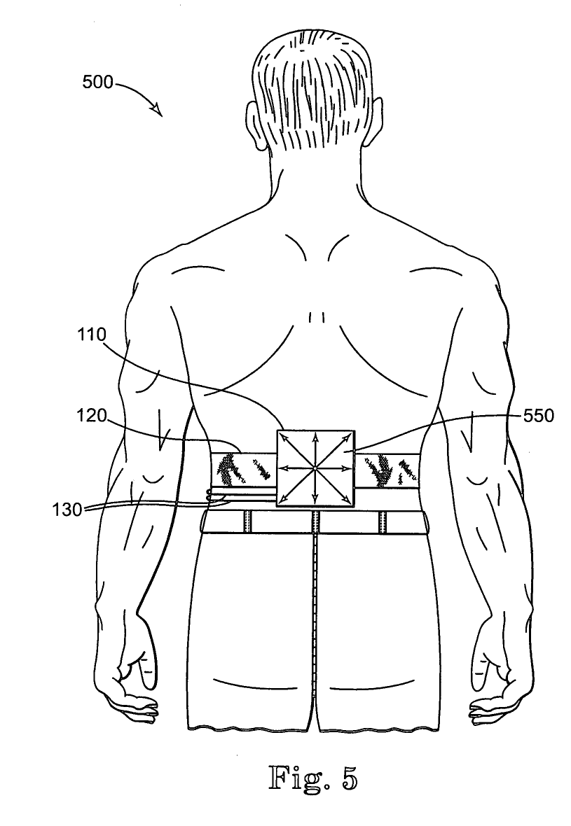

Another embodiment of transcutaneous electrical nerve stimulation is depicted in FIG. 5 showing one embodiment of the stimulation pad 110 secured by a belt-like device 120 as before. In this embodiment, energizing individual electrodes in the matrix array may allow for a traveling wave sensation for the patient such as the radial burst motion 550 depicted in FIG. 5.

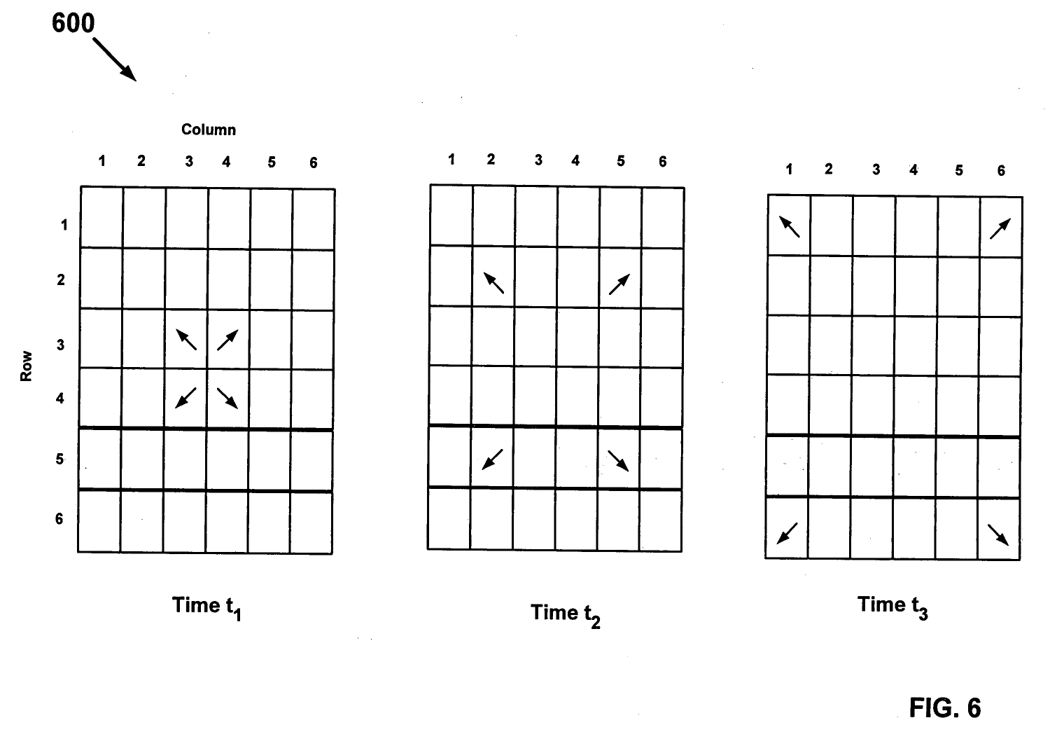

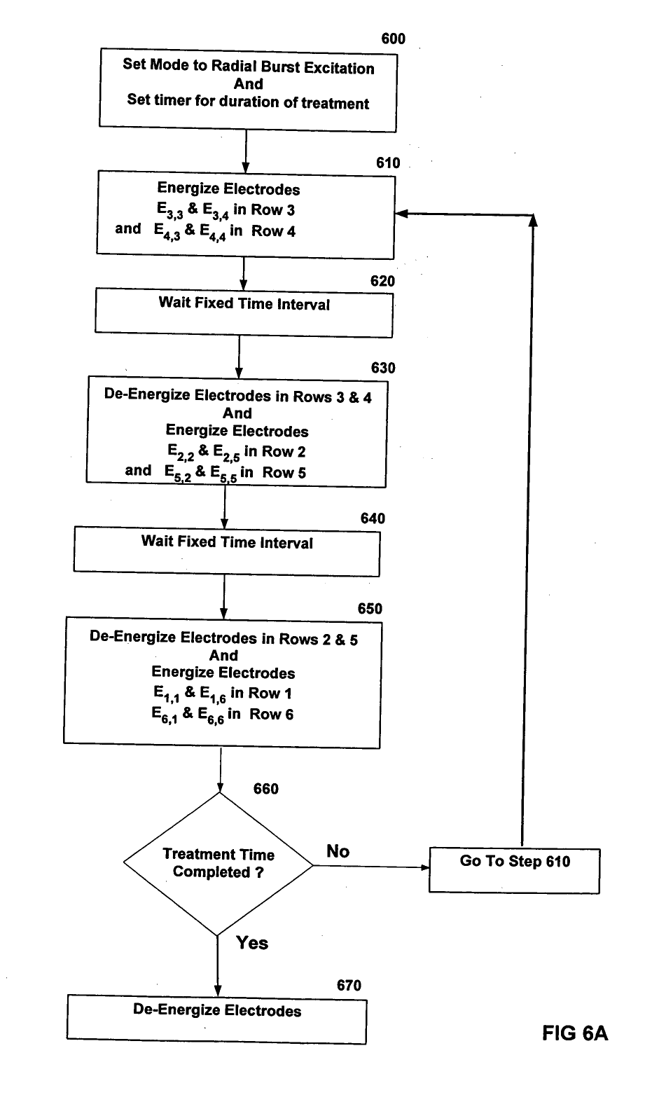

FIG. 6 shows one embodiment of the matrix array of electrodes 600 capable of generating the radial burst motion 550 mentioned above. In this embodiment, the matrix array of electrodes 600 is shown schematically as a 6-row by 6-column array of individual electrodes that may be individually energized. Again, the matrix size is for illustrative purposes only and is not limiting.

Each electrode in the two-dimensional array may be designated using standard matrix notation as E_{i,j}, representing the electrode in the i^{th} row and j^{th} column.

For example, E_{2,3} would represent the electrode in the 2^{nd} row and 3^{rd} column. As shown in FIG. 6 at time t₁, electrodes E_{3,3} & E_{3,4} in Row 3 and E_{4,3} & E_{4,4} in Row 4 may be energized by generating a localized transcutaneous electrical nerve stimulation near the central region of the stimulation pad 110.

A short time later at time t₂, electrodes E_{3,3}, E_{3,4}, E_{4,3} & E_{4,4} may be de-energized substantially synchronous with the energizing of electrodes E_{2,2} & E_{2,5} in row 2 and E_{5,2} & E_{5,5} in row 5, this localized transition may give the patient a sensation that the TENS signal is radiating (traveling) outward in the radial direction. And finally, a short time later at time t₃, electrodes E_{1,1} & E_{1,6} in row 1 and electrodes E_{6,1} & E_{6,6} in row 6, may be energized reinforcing the sensation that the TENS signal is traveling further away from the stimulation pad's 110 central region. The above spatial (electrode matrix stimulation) and temporal (at repetitive times t₁, t₂, and t₃) may be repeated over-and-over again to yield a soothing massage-like traveling wave sensation along with the transcutaneous electrical nerve stimulation. The radial burst TENS signal outlined above may be pre-programmed in the controller and initiated by accessing the program menu either manually or via vocal commands as described above.

FIG. 6A depicts a functional block diagram representing the algorithm that may be embedded in the controller to execute the radial burst TENS program shown in FIG. 6. In block 600 the user may select the radial burst excitation mode and select an auto-stop time interval. The user may then actuate the start button and then electrodes E_{3,3} & E_{3,4} in row 3 and E_{4,3} & E_{4,4} in row 4 will be energized 610. The controller may be programmed to dwell a predetermined time interval (620) with the electrodes in rows 3 and 4 energized. Once the predetermined time interval has elapsed, the controller unit may initiate a command to de-energize electrodes in rows 3 and 4 while energizing electrodes E_{2,2} & E_{2,5} in row 2 and E_{5,2} & E_{5,5} in row 5 (630). As before, the controller's processer may be programmed to dwell a predetermined time interval (640) with electrodes in rows 2 and 5 energized. Once the predetermined time interval set in block 640 has elapsed, the controller may initiate a command to de-energize electrodes in rows 2 and 5 while energizing electrodes E_{1,1} & E_{1,6} in row 1 and electrodes E_{6,1} & E_{6,6} in row 6 (650). After energizing electrodes in rows 1 and 6 for a preset time interval, the controller may compare the program run time with the user set treatment time in block 660, and if the run time exceeds the user set treatment time, the controller unit 140 may terminate the session by de-energizing the electrodes (670), otherwise the program may loop back to block 610 and continue to run until eventually timing out 660.

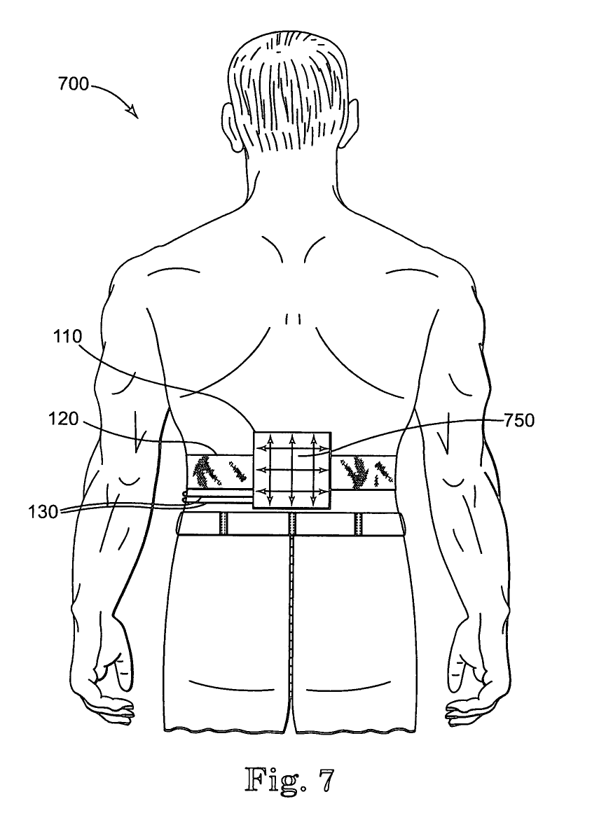

Another embodiment of a program, routine or algorithm that may be provided by the inventive device is illustrated in FIG. 7 wherein the stimulation pad 110 is secured by a belt-like fastener 120 as before. In this embodiment, energizing individual electrodes within the matrix array in a manner consistent with the method described above may allow for a traveling wave sensation for the user such as the simultaneous vertical and horizontal traveling waves of stimulation 750 illustrated in FIG. 7. The algorithm for realizing the traveling stimulation 750 of FIG. 7 may be obtained by simply combining the vertical algorithm described in connection with FIG. 2A with the horizontal algorithm outlined in FIG. 4A, though alternate methods will present themselves to those skilled in the art.

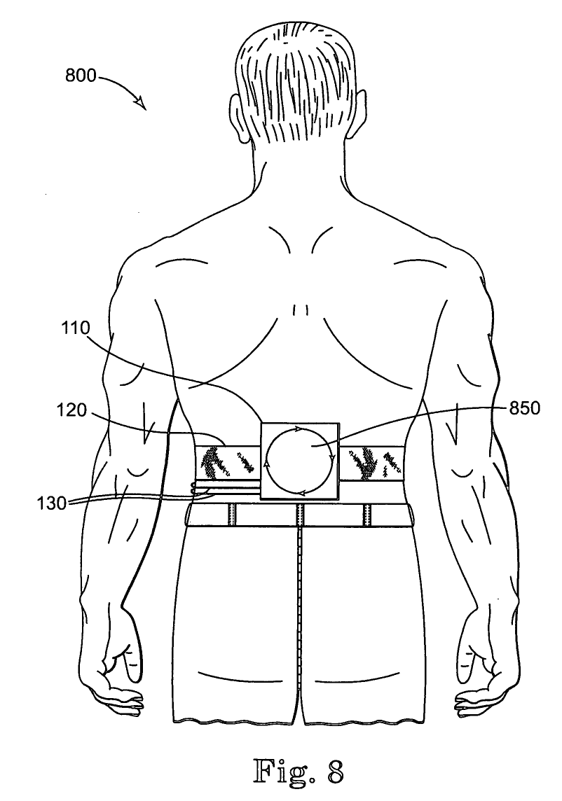

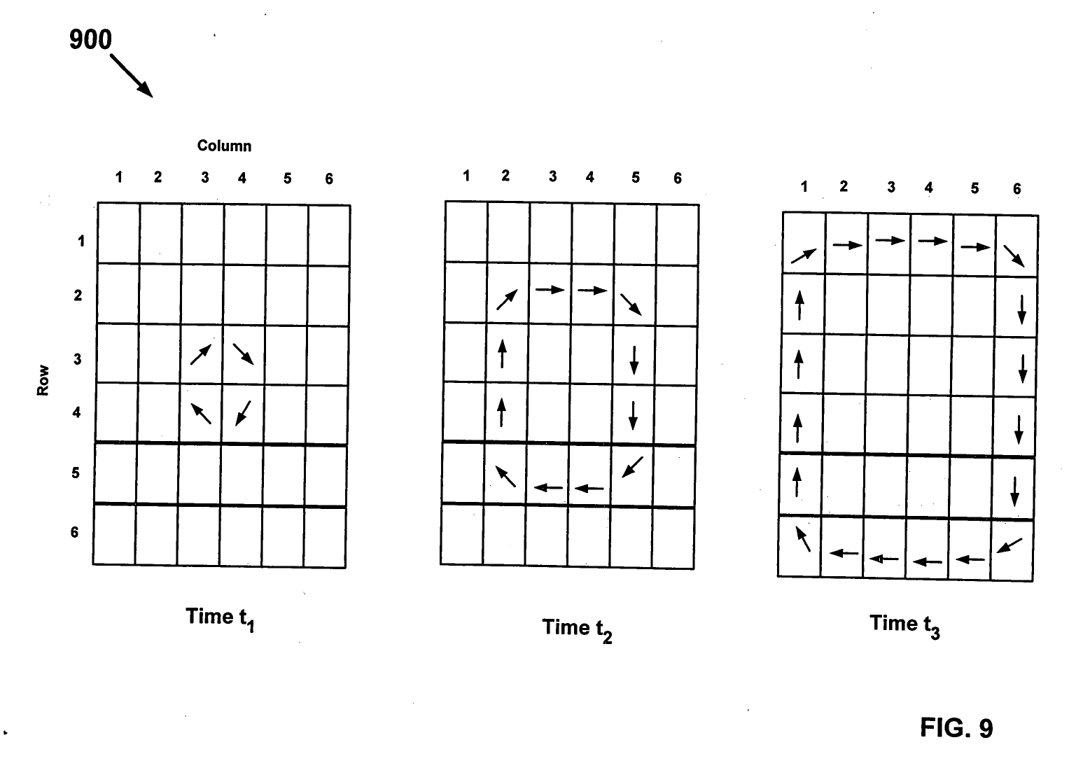

Yet another embodiment of TENS stimulation is provided in FIG. 8. This embodiment is provided to indicate that the matrix-based embodiment of the inventive device is not restricted to linear wave-like stimulation. Thus, FIG. 8 provides a stimulation pad with a circularly moving stimulation wave applied. In this embodiment, energizing individual electrodes within the matrix (or other pattern) array of electrodes may allow for a traveling wave sensation for the user such as the circular traveling wave-like stimulation 850 provided in FIG. 8. The circular traveling stimulation 850 may alternatively be expanded radially over time as shown in FIG. 9.

The timing diagram 900 in FIG. 9, and the circular traveling simulation with substantially constant radius of FIG. 8 may be accomplished in the same manner as described above in connection with FIGS. 2-7 and may be repeated to yield a repetitive radially expanding massaging effect. Again, numerous variations will readily present themselves to those skilled in the art, each such variation is within the scope of the invention.

Each of the above TENS stimulation routines, as well as all equivalents thereof, may be pre-programmed and stored in the controller's processor and memory and accessed through a keypad menu, voice activation or other means known to those skilled in the art. In addition to the ordered and repetitive routines, programs and algorithms outlined herein, the controller may have a pre-programmed routine that randomly energizes individual electrodes within the plurality of electrodes. Moreover, a program may cause the controller to switch or “hop” from one programmed routine to another after a period of time has passed. For example, 15 seconds of radial burst may be followed by 5 seconds of horizontal stimulation, etc.

Moreover, as described above, the user may either manually or via voice commands, locate the electrode actuation over a desired region of the lower back. In this mode, the user may elect to energize one or more electrode(s) at a time and manually scan through the plurality of electrode's matrix or patterned elements. Once the optimum location has been identified, the user may choose to initiate one of the pre-programmed, or manually programmed and stored, routines centered about the optimum location just located. For example, the user may choose the radial burst programmed pattern shown in FIG. 5 to initiate with the center at the optimum location.

Moreover, the controller may be amenable to controlling a plurality of stimulation pads either simultaneously or at alternate times. In this embodiment, the controller may then be used to provide individualized stimulation treatment to various members of a family at the same time, wherein each family member utilizes their own stimulation pad.

Alternatively, the controller may be programmed to recognize a plurality of individuals' stimulation preferences, including, inter alia, preferred stimulation routine(s), signal intensity and dwell time wherein the stimulation pad and controller is shared among the individuals.

As noted above, the present invention is applicable to medical devices and is believed to be particularly useful for treating lower back pains with trancutaneous electrical nerve stimulation with an adjunctive propagating motion that generates an additional massage-like therapeutic effect. The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

I claim:

1. A device for treating a user's lower back pain with transcutaneous electrical nerve stimulation, comprising:
a stimulation pad in operative contact with the user's lower back and comprising a plurality of electrodes arranged in a pattern wherein each electrode is individually manually and programmably actuable; and
a controller in wired or wireless communication with the plurality of electrodes and comprising a processor and memory, wherein the controller is capable of communicating programmed and manual actuation signals to the plurality of electrodes to create at least one synthetic traveling wave across the stimulation pad.

2. The device of claim 1, further comprising a transceiver in wired or wireless communication with the plurality of electrodes and wherein the controller is in wired or wireless communication with the transceiver, the controller further capable of communicating programmed and manual sequential actuation signals to the transceiver wherein the transceiver communicates the actuation signals to the plurality of electrodes to create at least one synthetic traveling wave across the stimulation pad.

3. The device of claim 1, wherein the plurality of electrodes comprise a flex circuit.

4. The device of claim 1, further comprising a fastener for holding the stimulation pad in contact with a user's lower back and supportably holding the transceiver.

5. The device of claim 1, wherein the position of the stimulation pad on the user's lower back is rotatably adjustable.

6. The device of claim 1, further comprising a visual display of the programmed and manual actuation signals in scaled correspondence to the location of the actuated electrodes within the stimulation pad to provide visual feedback to the user.

7. The device of claim 6, wherein the visual display is located on the controller.

8. The device of claim 6, wherein the visual display comprises a monitor in wired or wireless communication with the controller.

9. The device of claim 1, wherein the programmed and manual actuation of electrodes further comprises sequentially energizing and de-energizing more than one electrode within the electrode pattern to simulate at least one traveling wave of stimulation.

10. The device of claim 1, wherein the pattern of arranged electrodes further comprises a two-dimensional orthogonal electrode matrix array.

11. The device of claim 10, wherein the programmed and manual actuation of electrodes further comprises sequentially energizing and de-energizing more than one electrode within the electrode matrix array to simulate at least one traveling wave of stimulation.

12. The device of claim 1, wherein the programmed actuation of the plurality of electrodes further comprises programmed variation of electrode stimulation intensity and dwell time.

13. The device of claim 1, wherein the controller is capable of accepting programming by the user, wherein the controller stores the user programming to allow repeating of the user programming.

14. The device of claim 13, further comprising an electrode actuator on the controller, wherein the user programming comprises establishing a unique simulated traveling wave sequence wherein the user selectively actuates at least one of the plurality of electrodes with the electrode-actuating element to create the user-programming.

15. The device of claim 14, wherein the electrode actuator is selected from the group consisting of an electrostatic touchpad; a joystick; directional actuation buttons; and voice activated actuation.

16. The device of claim 15, wherein the electrostatic touchpad further comprises a scaled correspondence with the plurality of electrodes.

17. The device of claim 14, wherein the controller further comprises voice activation of the electrode actuator, whereby the user controls movement, intensity and/or dwell time of the electrode actuation through vocal commands.

18. The device of claim 1, further comprising voice-activated control of the controller.

19. The device of claim 18, further comprising voice-activated control of the programs, the electrode actuation intensity, and the dwell time.

20. The device of claim 1, wherein the stimulation pad further comprises heating elements.

21. The device of claim 2, wherein the stimulation pad further comprises heating elements.

22. The device of claim 1, the stimulation pad further comprising vibration elements.

23. The device of claim 2, the stimulation pad further comprising vibration elements.

24. A device for treating a user's lower back pain with transcutaneous electrical nerve stimulation, comprising:
a stimulation pad in operative contact with the user's lower back comprising a plurality of electrodes arranged in a pattern wherein each electrode is individually manually and programmably actuable and wherein the two-dimensional position of the stimulation pad is rotatably adjustable;
a transceiver in wired or wireless communication with the plurality of electrodes;
a fastener for holding the stimulation pad in contact with a user's lower back and supportably holding the transceiver;
a controller in wired or wireless communication with the transceiver and comprising a processor and memory, wherein the controller is capable of communicating programmed and manual sequential actuation signals to the transceiver and the transceiver communicates the actuation signals to the plurality of electrodes to create at least one synthetic traveling wave across the stimulation pad,
wherein the processor is capable of accepting programming by the user, wherein the processor's memory stores the user programming and further allows repeating of the user programming by the user, and wherein the programmed and manual actuation of electrodes further comprises sequentially energizing and de-energizing more than one electrode within the electrode matrix array to simulate at least one traveling wave of stimulation;
at least one electrode actuator on the controller, wherein the user programming comprises establishing a unique simulated traveling wave sequence wherein the user selectively actuates more than one of the plurality of electrodes with the at least one electrode actuator to create the user-programming; and
a visual display of the programmed and manual actuation signals in wired or wireless communication with the controller and further corresponding to the two-dimensional location of the actuated electrodes within the stimulation pad to provide visual feedback to the user.

25. A method for treating a user's lower back pain with transcutaneous electrical nerve stimulation, comprising:
providing a stimulation pad in operative contact with the user's lower back comprising a plurality of electrodes arranged in a pattern wherein each electrode is individually manually and programmably actuable;
providing a controller in wired or wireless communication with the plurality of electrodes and comprising a processor and memory, wherein the controller is capable of communicating programmed actuation signals to the plurality of electrodes to create at least one synthetic traveling wave across the stimulation pad; and
executing at least one electrode actuation program to create at least one synthetic traveling wave across the stimulation pad.

26. The method of claim 25, further comprising manually generating at least one electrode actuation signal and actuating at least one of the plurality of electrodes in response to the at least one manually generated electrode actuation signal.

27. The method of claim 25, further comprising providing a transceiver in wired or wireless communication with the plurality of electrodes and with the controller, wherein the transceiver receives the actuation signals from the controller and further transmits the actuation signals to the plurality of electrodes.

28. The method of claim 26, further comprising providing a transceiver in wired or wireless communication with the plurality of electrodes and with the controller, wherein the transceiver receives the actuation signals from the controller and further transmits the actuation signals to the plurality of electrodes.

29. The method of claim 25, further comprising using a visual display to provide feedback to the user regarding the electrode actuation location.

30. The method of claim 26, further comprising using a visual display to provide feedback to the user regarding the electrode actuation location.

31. The method of claim 25, further comprising instructing the controller to run in random mode comprising randomly modifying the variables selected from the list consisting of the electrode stimulation programs; electrode actuation intensity; and dwell time.

32. The method of claim 25, further comprising using voice-activated commands for selection of the at least one program to execute, selection of electrode actuation intensity, and/or selection of dwell time.

33. The method of claim 32, further comprising using voice-activated commands to modify the electrode actuation intensity and/or dwell time of the at least one selected program.

34. The method of claim 26, further comprising manually locating the electrode actuation over a specific region of the lower back.

35. The method of claim 34, further comprising using a manual electrode actuator to locate the electrode actuation over a specific region of the lower back.

36. The method of claim 35, further comprising using an electrostatic touchpad to locate the electrode actuation over a specific region of the lower back.

37. The method of claim 35, further comprising manually creating an electrode actuation routine using the manual electrode actuator.

38. The method of claim 26, further comprising:
manually generating a stimulation routine comprising a sequence of manually generated electrode actuation with a manual electrode actuator;
programming the stimulation routine in the controller;
storing the programmed routine; and
running the routine.

39. The method of claim 26, further comprising using voice commands to locate the electrode actuation over a specific region of the lower back.

40. The method of claim 39, further comprising using voice commands to select a program to operate wherein the program is substantially centered around the located electrode actuation of the specific lower back region.

41. The method of claim 26, further comprising:
programming a routine generated by voice-activated electrode actuation;
storing the routine; and
executing the routine.

42. The method of claim 41, further comprising using voice-activated commands to modify the electrode actuation intensity and/or dwell time of the programmed routine.

43. The method of claim 25, further comprising programming the controller for a plurality of users' preferences.

44. The method of claim 43, further comprising substantially simultaneously controlling a plurality of stimulation pads using one controller.
